(19) United States
(12) Patent Application Publication
(10) Pub. No.: US 2011/0158910 A1
(43) Pub. Date: Jun. 30, 2011

(54) MICROPARTICLES COMPRISING POLYMERS WITH THIOESTER BONDS

(76) Inventors: Aylvin Jorge Angelo Athanasius Dias, Maastricht (NL); Mark Johannes Boerakker, Eindhoven (NL); Jerome Lebouille, Munsterbilzen (BE); Tessa Kockelkoren, Beek (NL); Audrey Petit, Gionville (FR)

(21) Appl. No.: 12/989,466

(22) PCT Filed: Apr. 27, 2009
(86) PCT No.: PCT/EP2009/055086

§ 371 Date: Feb. 9, 2011

(30) Foreign Application Priority Data
Apr. 25, 2008 (EP) 08007983.3

(51) Int. Cl. A61K 49/00; C08G 75/00; A61K 47/34; A61K 38/02; A61K 39/00; A61K 31/7088; A61P 3/02; A61P 37/04; B32B 1/00; B82Y 5/00

(52) U.S. Cl. 424/9.1; 528/373; 514/772.3; 514/1.1; 424/184.1; 514/44 R; 428/402; 428/407; 977/773

(57) ABSTRACT

The present invention relates to particles suitable for delivery of active agents comprising a polymer containing thioester bonds which are obtained via the reaction of a thioic acid functionality and an unsaturated group. The polymer may be is linear, branched or crosslinked. The particles have an average diameter in the range of 10 nm to 1000 μm, preferably in the range of 100 nm-100 μm. The particles may comprise an active agent selected from the group of nutrients, pharmaceuticals, proteins and peptides, vaccines, genetic materials, diagnostic agents or imaging agents. The present invention further relates to the use of the particles in dermatology, muscular skeletal, oncology, in vascular applications, in orthopaedics, in ophthamology, spinal, intestinal, pulmonary, nasal, or auricular.

The invention relates to particles comprising polymers with thioester bonds, and a method of preparing such particles as well as the use of the particles in medical field.

Polymers comprising thioester bonds are known in the art and for example disclosed in US2002/071822. US2002/071822 describes polythioester polymers which are synthesised by a polycondensation reaction and comprise a backbone containing for example a thio-ester linkage, a biologically active compound and a hydrocarbon linking group. The biologically active compound is part of the polymer backbone and releases upon hydrolysis of the polymer. The properties of the polymer and hence of the device derived from it are directly related to the drug as the drug is part of the polymer backbone. Changing polymer properties is to a certain extent limited by the properties of the drug. This application also describes a synthetic approach that relies on a covalent bond between the polymer and the drug. This creates a number of limitations such as the polymer has to undergo hydrolysis to release the drug in the pure form. Thus there is a risk that partial polymer drug fragments are released or made bio available. The resultant drug fragments have to be understood in terms their half-life and bio distribution. The reformulation of a drug to modify its drug release would involve a synthetic step as opposed to formulating approach where release could be tailored via formulation with typical excipients used.

It is further disclosed that particles may be manufactured from the polythioesters. A disadvantage is however that the active agent is present in the polythioester backbone from which particles are produced. As a consequence the active agent is released upon hydrolysis of the polymer which results in the release of active agent but also in the degradation of the polymer at the same time. It is thus not possible to separate drug release from degradation, therefore it is also not possible to prepare particles where the drug release is purely based on diffusion and does not require degradation. Moreover not every drug permits to be covalently bound to the polymer backbone. A further disadvantage is that it is not possible to separate the drug loading and the microparticle manufacturing. In some cases it would be desirable to be able to provide particles which can be loaded with active agents after polymerisation, for instance because it would allow up-scaling of the preparation process of the particles to provide a large batch of the particles, of which—if desired—different portions can be loaded with different active agents, in useful quantities for a specific purpose. In other cases it would be desirable to provide particles which can be loaded after polymerisation because the active agent to be released from the particles may be detrimentally affected, e.g. degraded, denaturated or otherwise inactivated, during the preparation of the particles. A still further disadvantage is that upon degradation of the polymers, fragments may become available that contain the active ingredient as well as the hydrocarbon linker, the combination of which may adversely affect the therapeutic properties and/or mobility of the drug.

Accordingly it is the object of the present invention to overcome the above mentioned disadvantages and to provide particles suitable for delivery of active agents that can serve at least as an alternative to known particles.

It is the object of the present invention to provide particles comprising thioester polymers and an active agent wherein the polymer degradation, drug loading and drug release can be adjusted easily because the drug or active agent is not covalently bound to the polymer backbone.

It is a further object of the present invention to provide a particle comprising a thioester polymer that can adequately be loaded with an active agent during microparticle formation and/or after the microparticle has been prepared.

It is a still further object of the present invention to provide a microparticle being efficiently loadable with active agents.

It is a still further object of the present invention to provide polythioester particles where varied degradation time is achieved by changing the molecular weight of the polymer without substantially changing the composition, even though this always remains an alternative option. This is a significant benefit since changing the composition of the polymer effectively involves changing the polarity which thereby affects the polymer drug compatibility.

The object of the present invention is achieved in providing particles suitable for delivery of active agents comprising a polymer containing thioester bonds which are obtained via the reaction of a thioic acid functionality and an unsaturated group.

It has been found that particles made of these polythioesters provide several advantages over the above described particles such as more control over degradation, the option to separate polymer degradation from drug release, the option to load drugs which can not be covalently bound to the polymer. The particles of the present invention are efficiently loadable with active agents during microparticle formation and/or after the microparticles have been prepared.

Moreover it has been found that the particles according to the present invention seem to have a high resistance against aggressive processing conditions. Under aggressive processing conditions it is understood a condition that causes the particle to be subjected to a physical shock, such as a (fast) change in temperature for example a change of at least 1° C. per sec.—as happens in a freeze drying process or a sudden change in pressure, for example (repeated) pressurization and/or depressurization. For example in a pellet making machine use is made of a pressure of 0.5 T per cm² per sec.

The polythioesters used in the present invention are disclosed in WO-A-2007028612. Particles prepared from the polythioesters are not disclosed nor the advantages of the particles according to the present invention.

The polythioesters used in the present invention are obtained via the addition polymerisation reaction of a thioic acid functionality and an unsaturated group. Addition polymerization allows the preparation of polythioesters without the need for a polymerization catalyst or initiator. This is very useful in biodegradable polymers for medical and food applications since often the initiator fragments are materials that are not naturally metabolized or found in the body. The use of these polythioesters allows one to avoid any additional testing to determine the biological/metabolic fate of the initiator molecules.

In a preferred embodiment the particles comprise a polymer containing thioester bonds which are obtained via the reaction of a component X comprising at least one ethylenically unsaturated group with a component Y comprising at least two thioic acids, wherein X and/or Y is a low molecular fragment, an oligomer or a polymer and whereby at least one of X or Y is an oligomer or polymer allowing the components to form a polymer with at least two thioester bonds

Component X is presented by structural formula 1

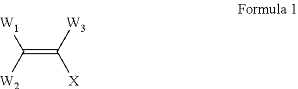

Wherein W1, W2 and W3 may be selected from the group consisting of C, H, O, N, S, P, alkyl, aryl, ester and ether. Preferably W1, W2 and W3 are hydrogen.

Component Y is presented by formula 2

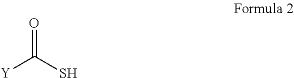

The method for the synthesis of the polymers containing thioester bonds requires the reaction of components X and Y. Such reaction, which may be a polymerisation, may be induced by light, in particular UV light, but may also be induced by heat such as body heat, with the help of an initiator such as AIBN, or occur spontaneously. When light, in particular UV, is used for the reaction, this may require the presence of a photoinitiator.

In formula 1 and 2, X and Y can be chemically diverse, they may be both degradable partially degradable or non degradable. This is often utilised where an additional property is required. X and/or Y are preferably degradable more preferably biodegradable, even more preferably metabolizable. X and Y may be based on the same oligomer or polymer, however, when they are based on different oligomers or polymers, the properties of the resulting particles comprising the polymer containing thioester bonds and the distribution of active agents such as drugs may be controlled more effectively and the reaction can be steered in a more controllable way. X and Y may also be based on a low molecular fragment which can be the same or different fragment.

X and Y may vary in molecular weight depending upon which properties are desired for the resulting polymer and particles made thereof. More particularly, the molecular weight of X and Y may range from about 28 Da to more than about 50000 Da. Prior to formation of the polymers and the particles, X and Y are synthesized to include thioic acid groups or ethylenically unsaturated groups such that they can participate in thioic-ene polymerisation. For use in in situ applications, the X and Y are preferably of higher molecular weight to limit migratibility of any unreacted materials

In the case of degradable polythioesters, X and/or Y can be selected from poly(lactide) (PLA), polyglycolide (PGA), co-oligomers or copolymers of PLA and PGA (PLGA), poly(anhydrides), poly(trimethylenecarbonates), poly(orthoesters), poly(dioxanones), poly(ε-caprolactones) (PCL), poly(urethanes), polyanhydrides, poly (hydroxy acids), polycarbonates, polyaminocarbonates, polyphosphazenes, poly(propylene)fumarates, polyesteramides, polyoxaesters, poly(maleic acids), polyacetals, polyketals, starch, and natural polymers such as polypeptides, polyhydroxyalkanoates, fibrin, chitin, chitosan, polysaccharides or carbohydrates such as polysucrose, hyaluronic acid, dextran and similar derivatives thereof, heparan sulfate, chondroitin sulfate, heparin, or alginate, and proteins such as gelatin, collagen, albumin, or ovalbumin, or co-oligomers or copolymers, or blends thereof.

In a particular preferred embodiment, X and/or Y are selected from poly(lactide) (PLA), poly(anhydrides), poly(trimethylenecarbonates, poly(dioxanones), poly(ε-caprolactones) (PCL), poly(lactide-co-glycolide) or co-oligomers or copolymers or blends thereof.

In the case where non degradable polythioesters are required for an additional property like hydrophilicity, hydrophobicity or mechanical strength the polymers, X and/or Y may be selected from the group consisting of poly (vinyl alcohol) (PVA), poly (ethylene oxide), poly (ethylene oxide)-co-poly(propylene oxide) block co-oligomers or copolymers (poloxamers, meroxapols), poloxamines, poly(urethanes), poly((polyethyleneoxide)-co-poly(butyleneterephtalate)), poly (vinyl pyrrolidone), poly (ethyl oxazoline), carboxymethyl cellulose, hydroxyalkylated celluloses such as hydroxyethyl cellulose and methylhydroxypropyl cellulose.

Particularly good amphiphilic behaviour can be achieved when X and/or Y. are selected from the group consisting of poly (ethylene oxide)-co-poly(propylene oxide), poloxamers, poloxamines, meroxapols.

Good mechanical strength may be achieved if polyurethanes are used as X and/or Y.

Particularly good hydrophilicity may be achieved if X and/or Y. are selected from the group consisting of poly (vinyl pyrrolidone) and poly (ethyl oxazoline).

The ethylenically unsaturated group as present in component X may be selected from a group consisting of vinyl, alkyne, alkene, vinyl ether, vinyl sulphones, vinylphosphates, allyl, acrylate, acrylamide, fumarate, maleate, itaconate, citraconate, mesaconate, methacrylate, maleimide, isoprene, and norbornene and derivatives thereof such as esters and amides.

The ethylenically unsaturated group is preferably chosen from the group consisting of vinyl, allyl, acrylate or fumarate.

Examples of component Y are dithio adipic acid (DTAA), tris[(6-oxo-6-sulfanylhexanoyl)oxy]poly(lactide-co-glycolide)2000 (PLGTTA), α,ω-bis[(6-oxo-6-sulfanylhexanoyl)oxy]poly(lactide-co-glycolide)1300 (PLGDTA) or 6-{2,3-bis[(6-oxo-6-sulfanylhexanoyl)oxy]propoxy}-6-oxohexanethioic S-acid (GTTA).

As used in this application, the term “oligomer” in particular means a molecule essentially consisting of a small plurality of units derived, actually or conceptually, from molecules of lower relative molecular mass. It is to be noted that a molecule is regarded as having an intermediate relative molecular mass if it has properties which vary significantly with the removal of one or a few of the units. It is also to be noted that, if a part or the whole of the molecule has an intermediate relative molecular mass and essentially comprises a small plurality of the units derived, actually or conceptually, from molecules of lower relative molecular mass, it may be described as oligomeric, or by oligomer used adjectivally. In general, oligomers have a molecular weight of more than 200 Da, such as more than 400, 800, 1000, 1200, 2000, 3000, or more than 4000 Da. The upper limit is defined by what is defined as the lower limit for the mass of polymers (see next paragraph).

Accordingly the term “polymer” denotes a structure that essentially comprises a multiple repetition of units derived, actually or conceptually, from molecules of low relative molecular mass. Such polymers may include branched polymers or linear polymers. It is to be noted that in many cases, especially for synthetic polymers, a molecule can be regarded as having a high relative molecular mass if the addition or removal of one or a few of the units has a negligible effect on the molecular properties. This statement fails in the case of certain macromolecules for which the properties may be critically dependant on fine details of the molecular structure. It is also to be noted that, if a part or the whole of the molecule has a high relative molecular mass and essentially comprises the multiple repetition of units derived, actually or conceptually, from molecules of low relative molecular mass, it may be described as either macromolecular or polymeric, or by polymer used adjectivally. In general, polymers have a molecular weight of more than 8000 Da, such as more than 10,000, 12,000, 15,000, 25,000, 40,000, 100,000 or more than 1,000,000 Da.

The term low molecular fragment means a molecule with a Mw below 1000 Da such as an aliphatic, cycloaliphatic or aromatic molecule with for example from 2-18 C atoms.

The particles according to the present invention may comprise a linear, branched or crosslinked polymer containing thioester bonds.

In case of linear polymers it may be advantageous that component X comprises a maximum of 2 ethylenically unsaturated groups and that component Y comprises a maximum of 2 thioic acid groups. The minimum average ethylenically unsaturated groups and thioic acid groups per component is advantageously larger than 1.2. In case of linear polymers it is of importance that the polymer formed has a melting temperature above 40 degrees centigrade because it will be a solid below this temperature. These linear polymers are the most preferred ones.

In case of crosslinked polymers or networks, it is required that component X comprises at least 2 ethylenically unsaturated groups and that component Y comprises at least 2 thioic acid groups and that the number of ethylenically unsaturated groups plus thioic acid groups is more than 4.

The properties of the polythioesters may be influenced by the degree of cross-linking. This may be achieved by choosing appropriate chain lengths of the components X and Y. Alternatively, the degree of cross-linking may be influenced by choosing an appropriate number of ethylenically unsaturated groups in component X and/or thioic acid groups in component Y. In another alternative the degree of cross-linking may be influenced by preventing the polymerization to go to completion, i.e. by preventing the highest degree of reaction to occur. Preferably, however, the reaction proceeds to the highest degree of reaction. A partial reaction may be especially desirable when it is required to have some residual reactive groups in the cross-linked matrix, for instance for modifications after cross-linking, such as attaching functional groups or covalent attachment to tissue or other biological material.

Cross-linking may be carried out in any suitable way known for cross-linking compounds comprising vinyl groups, in particular by thermal initiation (aided by a thermo initiator, such as a peroxide or an azo-initiator, e.g. azobisisobutyronitrile (AIBN), by photo-initiation (aided by a photo-initiator such as a Norrish type I or II initiator), by redox-initiation, or any (other) mechanism that generates radicals making use of a chemical compound and/or electromagnetic radiation. Examples of suitable crosslinkers are trimethylolpropane trimethacrylate, diethylene glycol dimethacrylate or hydroxyethylacrylate.

In case of particularly strong crosslinked polymers or networks, it is required that component X comprises at least 3 ethylenically unsaturated groups and/or that component Y comprises at least 3 thioic acid groups and that the number of ethylenically unsaturated groups plus thioic acid groups is more than 5.

In case of branched polymers or non-gelled polymers it is required that the composition comprising components X and Y fulfils the boundary conditions for compositions for branched, non-gelled polymers as reported by Durand and Bruneau (D. Durand, C.-M. Bruneau, Makromol. Chem. 1982, 183, 1007-1020 and in D. Durand, C.-M. Bruneau, The British Polymer Journal, 1979, 11, 194-198; D. Durand, C.-M. Bruneau, The British Polymer Journal 1981, 13, 33-40; D. Durand, C.-M. Bruneau, Polymer, 1982, 23, 69-72; D. Durand, C.-M. Bruneau, Makromol. Chem., 1982, 183, 1021-1035; D. Durand, C.-M. Bruneau, Polymer, 1983, 24, 587-591).

In a further preferred embodiment the polythioesters comprise fragments of formula 3 and/or formula 4.

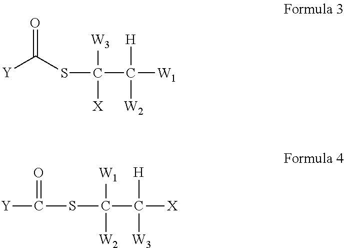

wherein
X and/or Y is a low molecular fragment, an oligomer or a polymer whereby at least one of X or Y is an oligomer or polymer and whereby
W1, W2 and W3 are selected from the group consisting of C, H, O, N, S, P, alkyl, aryl, ester and ether.
   It is preferred that W1, W2 and W3 are H.

The polythioester may also contain a fragment according to formula 5,

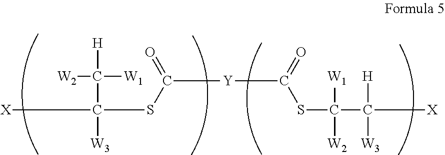

wherein
W1, W2 and W3 are selected from the group consisting of H, C, O, N, S, P, alkyl, aryl, ester and ether,
Y can be of a low molecular weight fragment, an oligomer or polymer, X can be the same or different low molecular weight fragment, oligomer or polymer whereby at least one of X or Y is an oligomer or polymer.
m and n are integers the sum of which indicates the number of thioester linkers connected to Y, wherein the sum of m and n is at least 2.

Depending on the particular kind of oligomer or polymer, chosen for X or Y, degradability of the particles may be influenced. For instance, particles manufactured from polymers containing non-degradable triethyleneglycol divinyl ether (TEGDVE) as component X will show lower degradation rates when compared to particles manufactured from polymers based on degradable component X containing ethylenically unsaturated groups, such as poly(lactide-co-glycolide)1200di(4-pentenoate) (PLGDP) or poly(lactide-co-glycolide)2600-tri(4-pentenoate) (PLGTP). Hydrophobic component poly(ε-caprolactone)2100di(4-pentenoate) (PCLDP) was designed to degrade over years.

The polymers containing the thioester bonds have the advantageous property that they can be degraded hydrolytically. When the components X and Y are also degradable or biodegradable, a polymer may be synthesized that can be degraded more completely with no residues left. When the components X and Y are even completely degradable or biodegradable, a polymer may be synthesized that can be degraded without leaving any residual components.

The particles of the present invention are suitable in medical field and in particular suitable as a delivery system for active agents such as drugs, diagnostic aids or imaging aids. The particles can also be used to fill a capsule or tube by using high pressure or may be compressed as a pellet, without substantially damaging the particles. It can also be used in injectable or spray-able form as a suspension in a free form or in an in-situ forming gel formulation. Furthermore, the particles can be incorporated in for example (rapid prototyped) scaffolds, coatings, patches, composite materials, gels, plasters or aerosols. The particles according to the present invention can be injected, sprayed, implanted or absorbed.

Particles have been defined and classified in various different ways depending on their specific structure, size, or composition, see e.g. Encyclopaedia of Controlled drug delivery Vol 2 M-Z Index, Chapter: Microencapsulation Wiley Interscience, starting at page 493, see in particular page 495 and 496.

As used in the present invention the term particles includes micro- or nanoscale particles which are typically composed of solid or semi-solid materials and which are capable of carrying an active agent. Typically, the average diameter of the particles ranges from 10 nm to 1000 μm, preferably from 10 nm to 500 μm, more preferably from 10 nm to 100 μm. In fact the most preferred average diameter depends on the intended use.

Microparticles according to the present invention typically have an average diameter ranging from 1 μm to 1000 μm. In case that the particles are intended for use as an injectable drug delivery system, in particular as an intravascular drug delivery system, an average diameter of up to 10 μm, in particular in the range of 1 to 10 μm, preferably in the range of 1-5 μm may be desired.

Nanoparticles according to the present invention typically have an average diameter below 1000 nm, for example ranging from 10 nm-999 nm. Preferably ranging from 20-800 nm, more preferably from 30-500 nm. For intravascular purposes, the average diameter is preferably ranging from 100-300 nm, for intracellular purposes the average diameter is preferably ranging from 10-100 nm. In other applications, other dimensions may be desirable, for instance an average diameter in the range of 10 nm to 500 nm, preferably in the range from 10 nm to 300 nm.

In particular, the particle diameter as used herein is the Z-average diameter as determinable by a Malvern Zetasizer NanoZS Dynamic lightscattering (Malvern Instrument Inc.), making use of an ASTM certified polymer latex size standard of 60 nm as a control. Z-Average diameters are calculated directly from the correlation function measured and therefore do not depend on the input of physical properties of the particles. Next to particle size analysis and if the particles are non analyzable by light scattering, because of their optical properties, then scanning electron microscopy (SEM) or transmission electron microscopy (TEM) can be used.

Microparticles in general have an average diameter larger than 1 μm. In particular, the particle diameter used is the D50 or median value of a volume-based size distribution (model independent) as determinable by a Coulter LS-230 Series Laser diffraction particle sizer, making use of a finely powdered UHMwPE powder (70-150 μm) as a control sample. The particle size distribution is calculated from diffraction data assuming a Fraunhofer-model (no corrections for refractive indexes of materials).

Several types of particle structures can be prepared according to the present invention. These include substantially homogenous structures, including nano- and microparticles and the like. However in case that more than one active agent has to be released or in case that one or more functionality is needed it is preferred that the particles are provided with a structure comprising an inner core and an outer shell. A core/shell structure enables more multiple mode of action for example in drug delivery of incompatible compounds or in imaging. The shell can be applied after formation of the core using a spray drier. The core and the shell may comprise the same or different polymers containing thioester bonds with different active agents. In this case it is possible to release the active agents at different rates. It is also possible that the active agent is only present in the core and that the shell is composed of the hydrolysable polymer containing the thioester bonds.

In a further embodiment the particles may comprise a core comprising the polymers containing thioester bonds and a shell comprising a magnetic or magnetisable material.

In still a further embodiment, the particles may comprise a magnetic or magnetisable core and a shell comprising the polymers containing thioester bonds. Suitable magnetic or magnetisable materials are known in the art. Such particles may be useful for the capability to be attracted by objects comprising metal, in particular steel, for instance an implanted object such as a graft or a stent. Such particles may further be useful for purification or for analytical purposes.

In a still further embodiment, the particles may be imageable by a specific technique. Suitable imaging techniques are MRI, CT, X-ray. The imaging agent can be incorporated inside the particles or coupled onto their surface. Such particles may be useful to visualize how the particles migrate, for instance in the blood or in cells. A suitable imaging agent is for example gadolinium.

The particles according to the present invention may carry one or more active agents or drugs. An active agent may be more or less homogeneously dispersed within the particles or within the microparticle core. The active agent may also be located within the microparticle shell.

In particular, the active agent may be selected from the group of nutrients, pharmaceuticals, proteins and peptides, vaccines, genetic materials, (such as polynucleotides, oligonucleotides, plasmids, DNA and RNA), diagnostic agents, and imaging agents. The active agent, such as an active pharmacologic ingredient (API), may demonstrate any kind of activity, depending on the intended use.

The active agent may be capable of stimulating or suppressing a biological response. The active agent may for example be chosen from growth factors (VEGF, FGF, MCP-1, PIGF, PDGF, TGF-B, growth factor inhibiting compounds such as, antibiotics (for instance penicillin's such as B-lactams, chloramphenicol), non steroidal anti-inflammatory drugs (NSAIDs) including drugs based on Salicylates like Acetylsalicylic acid, Aspirin, Amoxiprin, Benorylate/Benorilate, Choline magnesium salicylate, Diflunisal, Ethenzamide, Faislamine, Methyl salicylate, Magnesium salicylate, Salicyl salicylate, Salicylamide, drugs based on Arylalkanoic acid such as Diclofenac, Aceclofenac, drugs based on 2-Arylpropionic acids (profens) such as Ibuprofen or Alminoprofen, drugs based on N-Arylanthranilic acids (fenamic acids) such as Mefenamic acid, Flufenamic acid, Ampyrone and COX-2 inhibitors such as Celecoxib, Eetoricoxib, Lumiracoxib, TGA cancelled registration such as Parecoxib Rofecoxib, Valdecoxib and Sulphonanilides but also other anti-inflammatory compounds, antithrombogenic compounds, anti-claudication drugs, anti-arrhythmic drugs, anti-atherosclerotic drugs, antihistamines, cancer drugs, vascular drugs, ophthalmic drugs, amino acids, vitamins, hormones, neurotransmitters, neurohormones, enzymes, signalling molecules and psychoactive medicaments.

Examples of specific active agents or drugs are neurological drugs (amphetamine, methylphenidate), alpha1 adrenoceptor antagonist (prazosin, terazosin, doxazosin, ketenserin, urapidil), alpha2 blockers (arginine, nitroglycerin), hypotensive (clonidine, methyldopa, moxonidine, hydralazine minoxidil), bradykinin, angiotensin receptor blockers (benazepril, captopril, cilazepril, enalapril, fosinopril, lisinopril, perindopril, quinapril, ramipril, trandolapril, zofenopril), angiotensin-1 blockers (candesartan, eprosartan, irbesartan, losartan, telmisartan, valsartan), endopeptidase (omapatrilate), beta2 agonists (acebutolol, atenolol, bisoprolol, celiprolol, esmodol, metoprolol, nebivolol, betaxolol), beta2 blockers (carvedilol, labetalol, oxprenolol, pindolol, propanolol) diuretic actives (chlortalidon, chlorothiazide, epitizide, hydrochlorthiazide, indapamide, amiloride, triamterene), calcium channel blockers (amlodipin, barnidipin, diltiazem, felodipin, isradipin, lacidipin, lercanidipin, nicardipin, nifedipin, nimodipin, nitrendipin, verapamil), anti arrthymic active (amiodarone, solatol, atenolol, diclofenac, enalapril, flecamide) or ciprofloxacin, latanoprost, flucloxacillin, rapamycin and analogues and limus derivatives, paclitaxel, taxol, cyclosporine, heparin, corticosteroids, triamcinolone.acetonide, dexamethasone, fluocinolone acetonide), anti-angiogenic (iRNA, VEGF antagonists: bevacizumab, ranibizumab, pegaptanib), growth factor, zinc finger transcription factor, triclosan, insulin, salbutamol, oestrogen, norcantharidin, microlidil analogues, prostaglandins, statins, chondroitinase, diketopiperazines, macrocycli compounds, neuregulins, osteopontin, alkaloids, immuno suppressants, antibodies, avidin, biotin, clonazepam.

The active agent can be delivered for local delivery or as pre or post surgical therapies for the management of pain, osteomyelitis, osteosarcoma, joint infection, macular degeneration, diabetic eye, diabetes mellitus, psoriasis, ulcers, atherosclerosis, claudication, thrombosis viral infection, cancer or in the treatment of hernia.

In accordance with the present invention, if an active agent is present, the concentration of one or more active agents in the particles, is preferably at least 5 wt. %, based on the total weight of the particles, in particular at least 10 wt. %, more in particular at least 20 wt. %. The concentration may be up to 90 wt. %, up to 70 wt. %, up to 50 wt. % or up to 30 wt. %, as desired.

The fields wherein particles according to the present invention can be used include dermatology, muscular skeletal, oncology, vascular, orthopedics, ophthalmic, spinal, intestinal, pulmonary, nasal, or auricular.

Besides in a pharmaceutical application, particles according to the invention may inter alia be used in an agricultural application. In particular, such particles may comprise a pesticide or a plant-nutrient.

It is also possible to functionalise at least the surface of the particles by providing at least the surface with a functional group, in particular with a signalling molecule, an enzyme or a receptor molecule, such as an antibody. The receptor molecule may for instance be a receptor molecule for a component of interest, which is to be purified or detected, e.g. as part of a diagnostic test, making use of the particles of the present invention. Suitable functionalisation methods may be based on a method known in the art. In particular, the receptor molecule may be bound to the polymer of which the particles or nanoparticles are composed.

To couple a target functional moiety comprising an amide group N-hydroxysuccinimide (NHS) may be used. In particular NHS may be coupled to the particles if the particles comprise a polyalkylene glycol moiety, such as a PEG moiety.

A target functional moiety may also comprise an —SH group, for example a cysteine residue which may be coupled to the particles by first reacting the particles with vinyl sulfone. In particular vinyl sulfone may be coupled to the particles if the particles comprise a polyalkylene glycol moiety, such as a PEG moiety. Various other coupling agents are known, (See Fisher et. al. Journal of Controlled release 111 (2006) 135-144 and Kasturi et. al. Journal of Controlled release 113 (2006) 261-270.

In addition to the polymer containing thioester bonds the microparticles or nanoparticles according to the present invention may further comprise one or more other compounds selected from the group of polymers and cross-linkable or polymerisable compounds. The polymers may in particular be polymers such as described above. The crosslinkable or polymerisable compounds may in particular be compounds selected from the group of acrylic compounds and other olefinically unsaturated compounds, for example, vinyl ether, allylether, allylurethane, fumarate, maleate, itaconate or unsaturated acrylate units. Suitable unsaturated acrylates are, for example, unsaturated urethaneacrylates, unsaturated polyesteracrylates, unsaturated epoxyacrylates and unsaturated polyetheracrylates.

The other polymers or polymerisable compounds may be used to adjust a property of the particles, for example to tune the release profile of an active agent or to obtain a complete polymerization (i.e. no residual reactive unsaturated bonds that may be cytotoxic) or to narrow the size distribution of the microparticle.

Loading of the particles may be achieved by forming the particles in the presence of the active agent or thereafter. To achieve particles with a high amount of active agent, it is generally preferred to prepare the particles in the presence of the active agent. In particular in the case that the active agent is sensitive to the cross-linking or may adversely affect or interfere directly or indirectly with the cross-linking, it is preferred to load the particles with active agent after they have been formed. This can be achieved by contacting the particles with the active agent and allowing the agent to diffuse into the particles and/or adhere/adsorb to the surface thereof.

In principle particles may be prepared in a manner known in the art, provided that the polymers used in the prior art are (at least partially) replaced by the polymers containing the thioester bonds. The microparticle/nanoparticles of the present invention are preferably prepared by the steps of dissolving a polymer containing thioester bonds in at least one organic solvent, miscible or partially miscible with water, subsequently adding a drug and dissolving or dispersing it. The resulting organic solution is than added to an aqueous solution containing a surfactant or surface active agent and stirred. The organic solvent can be removed by evaporation or extraction. The particles are then dried to obtain drug-loaded particles. The choice of the organic solvents used in this process is dependent on the solubility of the drug or the active agent. It is possible to use a blend of organic solvents to improve the solubility of the active agent.

Another route to prepare the microparticles/nanoparticles according to the present invention comprising a drug is for example by dissolving the polymer in an organic solvent, miscible or partially miscible with water and adding it to an aqueous solution containing a surfactant or surface active agent. The resulting mixture is stirred and the polymer composition is crosslinked, upon which the organic solvent is removed by extraction or evaporation. After washing and drying the crosslinked particles, a drug molecule is added in an organic solvent followed by the evaporation of the solvent. Subsequently the particles are washed and dried to obtain drug-loaded particles and the organic solvent is evaporated.

It is however also possible to prepare the particles according to the present invention by the following method of dissolving the drug in an aqueous solution, adding it to an organic solution containing the polymer containing thioester bonds, mixing the resulting mixture, adding the resulting mixture to an aqueous solution containing a surfactant or a surface active agent and stirring it. Next removing the an organic solvent by evaporation or extraction and drying the particles.

In accordance with the invention it is possible to provide particles with one or more active agents with satisfactory encapsulation efficiency. (i.e. the amount of active agent in the particles, divided by the amount of active agent used). Depending upon the loading conditions, an efficiency of at least about 50%, at least about 75% or at least 90% or more is feasible.

The invention will now be illustrated by the following examples without being limited thereto.

### Materials and Methods

Nuclear Magnetic Resonance (NMR) experiments were performed on a Varian Inova 300 spectrometer.

Infrared experiments were performed on a Perkin Elmer Spectrum FT-IR Spectrometer 1760×, 1720×. The polymer samples were placed between two KBr tablets.

Size Exclusion Chromatography (SEC) was performed using a Waters 515 HPLC pump, a Waters 410 Differential Refractometer and a Servern Analytical SA6503 Programmable Absorbance Detector equipped with a Waters Styragel HR 2, 3 and 4 column at flow rate of 1 ml/min using tetrahydrofuran (THF) as the eluent. SEC data were obtained using the IR detector. The system was calibrated using narrow polystyrene standards (EasyCal PS2, from Polymer Laboratories, Heerlen).

LST 230 Series Laser Diffraction Particle size analyzer (Beckman Coulter) was used to measure size distribution of the particles. The standard was UHMwPE (0.02-0.04 μm).

A Leica DMLB microscope (magnitude ×50 to ×400) was used to analyse the morphology of the particles.

A Philips, CP SEM XL30 at an accelerating voltage of 5 and 10 kV, was used to examine the particles. The specimens were mounted in a SEM sample holder and a conductive Au-layer was applied (2*60 s, 20 mA).

A Malvern Zeta-sizer NanoZS Dynamic lightscattering (Malvern Instrument Inc.), was used to determine the Z-average diameter of the nanoparticles. This instrument uses an ASTM certified polymer latex size standard of 60 nm as a control. Z-Average diameters are calculated directly from the correlation function measured and therefore do not depend on the input of physical properties of the particles.

### EXAMPLE 1

### Synthesis of Polythioester without the Addition of a Polymerisation Catalyst

0.83 g (4.64 mmol) of dithio-adipic acid (DTAA) was added to 15 ml of freshly distilled dry THF. To this 1 equivalent of triethylene glycoldivinyl ether (0.938 g; 4.64 mmol) is added. The solution was heated to 80° C. under nitrogen for 12 hrs.

### EXAMPLE 2

### Synthesis of Polythioester with Polymerisation Catalyst (AIBN)

0.83 g (4.64 mmol) of dithio-adipic acid (DTAA) was added to 15 ml of freshly distilled dry toluene. To this 1 equivalent of triethylene glycoldivinyl ether (0.938 g; 4.64 mmol) and 0.4 mmol (0.07 g) of AIBN was added. The solution was heated to 80° C. under nitrogen for 12 hrs.

### EXAMPLE 3

### Synthesis of Polythioester with a Peroxide Initiator

0.83 g (4.64 mmol) of dithio-adipic acid (DTAA) was added to 15 ml of freshly distilled dry THF. To this 1 equivalent of triethylene glycoldivinyl ether (0.938 g; 4.64 mmol) and 0.4 mmol (0.01 g) of benzoyl peroxide was added. The solution was heated to 90° C. under nitrogen for 12 hrs.

### EXAMPLE 4

### Synthesis of a Polythioester with Polyester Macromer Building Blocks with Polymerisation Catalyst (AIBN) in Solution

0.83 g (4.64 mmol) of dithio-adipic acid (DTAA) was added to 100 ml of dry toluene, to this 1 equivalent (4.64 mmol; 46.4 g) of PLGA 75/25 10.000 diene was added. To this solution 0.13 g (0.8 mmol) of AIBN was added. The mixture was refluxed at 80° C. under nitrogen for 8 hrs.

The resultant polymer solution was precipitated in cold hexane.

### EXAMPLE 5

### Synthesis of a Polythioester with Polyester Macromer Building Blocks with Polymerisation Catalyst (Benzoyl Peroxide) in Solution

0.83 g (4.64 mmol) of dithio-adipic acid (DTAA) was added to 100 ml of dry THF, to this 1 equivalent (4.64 mmol; 46.4 g) of PLGA 75/25 10.000 diene was added. To this solution 0.05 g (0.2 mmol) of benzoyl peroxide was added. The mixture was refluxed at 90° C. under nitrogen for 8 hrs.

The resultant polymer solution was precipitated in cold hexane.

### EXAMPLE 6

### Synthesis of PLGA 10000 Diene

The degradable oligomer poly (lactide-co-glycolide) 10000di (4-pentenoate) was synthesized via poly (lactide-co-glycolide) 10000diol. Thereto, 38.69 g (265.80 mmol) of dl-lactide, 10.39 g (88.69 mmol) of glycolide and 0.5316 g (5.00 mmol) of diethyleneglycol were melted at 150° C. 500 μl of a hexane solution containing 15 mg of tindioctoate was added. The reaction was allowed to proceed for 24 h upon which the reaction mixture was cooled to room temperature to obtain the product. Yield: 98% as a slight yellow solid.

Next, poly(lactide-co-glycolide)10000diol (49 g, 49 mmol) was dissolved in THF (300 ml), triethylamine (1.22 g, 12 mmol) was added and the reaction mixture was cooled to 0° C. upon which pentenoylchloride (1.26 g, 11 mmol) was added and the temperature was maintained at 0° C. for 1 h. The mixture was left to stir at room temperature. Next, the reaction mixture was stirred for 20 min at 0° C. to precipitate the triethylamine hydrochloride salts formed during the reaction. The mixture was filtered and concentrated in vacuo. The residue was redissolved in chloroform and extracted with saturated aqueous NaCl solution and distilled water. The organic layer was dried over Na₂SO₄ and the solvent was removed under vacuum.

Yield 81% as an off-white solid.

### EXAMPLE 7

### Synthesis of PLGA Comprising at Least Two Thioester Bonds

Composition was prepared with equimolar ratios of PLGA10000diene and dithioic adipic acid 1 wt % of Darocure 1173 and ethyl acetate as a solvent (15 wt %). The composition was applied to a glass plate and exposed to UV-light (D-bulb, 15 J/cm²). The obtained polymer was dried in the oven to obtain an off-white solid.

### EXAMPLE 8

### Microparticle Preparation

730 mg of PLGA comprising two thioester bonds, as synthesized in example 7, was dissolved in 7 ml of a DMSO/ethylacetate mixture (10/90 v/v) and added to 21 ml of aqueous polyvinylalcohol solution (1 wt %) while stirring mechanically at 800 rpm.

Microparticles were obtained with a mean average diameter of 50 micrometer.

### EXAMPLE 9

### Microparticle Preparation

730 mg of PLGA comprising two thioester bonds, as synthesized in example 7, was dissolved in 7 ml of dichloromethane and added to 21 ml of aqueous polyvinylalcohol solution (1 wt %) while stirring mechanically at 800 rpm.

Microparticles were obtained with a mean average diameter of 100 micrometer.

### EXAMPLE 10

### Preparation of Microparticles Through Water in Oil in Water (W/O/W) Process

100 mg of polymer prepared from PLGA8000 diene and DTAA was dissolved in DCM (4 mL) and 11 mg of myoglobin was dissolved in 150 microliter of H₂O. When both were dissolved, both solutions were combined and vortexed at maximum speed for 30 seconds. Immediately after this step, the vortexed emulsion was added to 20 mL of 1% PVA solution and this was mechanically stirred at 800 rounds per minute for 4 hours. When the particle formation was complete, the w/o/w-emulsion was centrifuged 3× for 4 min at 3000 rpm and washed after each centrifugation step with H₂O. 50% of myoglobin was encapsulated in the microparticles. The particles had an average size of 70-80 micrometer.

### EXAMPLE 11

### Freeze-Drying of W/O/W Microparticles Based on PLGA Comprising at Least Two Thioester Bonds

For storage, the microparticles from example 10 were freeze dried using a Christ Alpha 1-2 LD Plus freeze dryer. The samples were first frozen in liquid nitrogen and the cap was replaced by a tissue to allow evaporation of the ice. The samples were placed in the freeze dryer overnight. The next morning, the samples were placed in the fridge at 4° C. SEM analysis showed that did not rupture or show any damage as a result of the freeze-drying process.

### Comparative Experiment A

Analogous microparticles, as described in example 11, were made from commercial PLGA RG502 instead of from PLGA comprising at least two thioester bonds. The SEM analysis showed ruptures after freeze-drying.

### EXAMPLE 12

### Preparation of Microspheres Through Water-in-Oil-in-Water (W/O/W) Process

100 mg of polymer prepared from PLGA8000 diene and diethyleneglycol-dithioic acid (DEGDTA) as shown in formula 6 was dissolved in dichloromethane (DCM) (4 mL) and 11 mg of myoglobin was dissolved in 150 microliter of H₂O. When both were dissolved, both solutions were combined and mixed at maximum speed for 30 seconds. Immediately after this step, the resulting emulsion was added to 20 mL of 1% PVA solution and this was mechanically stirred at 800 rounds per minute for 4 hours. When the particle formation was complete, the w/o/w-emulsion was centrifuged 3× for 4 min at 3000 rpm and washed after each centrifugation step with H₂O. 40% of myoglobin was encapsulated in the microparticles. The particles had an average size of 85 micrometer.

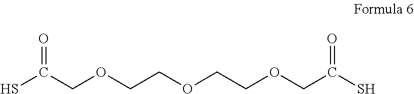

### EXAMPLE 13

### Nanosphere Preparation Based on PLGA Comprising at Least Two Thioester Bonds

A solution of 1.9 wt % PLGA comprising two thioester bonds, as synthesized in example 7, in acetone was injected into a solution of 0.5 wt % surfactant (Pluronic F127) in demi water. DLS of the nanoparticle suspension showed nanoparticles with an average diameter of 143 nm.

### EXAMPLE 14

### Preparation of Nanoparticles of PLGA Comprising at Least Two Thioester Bonds (PLGA-PTE) Including Drugs

PLGA comprising at least two thioester bonds (PLGA-PTE), as synthesised in example 7 has been used to prepare nanoparticles comprising Rapamycin (Rapa), Dexamethason (Dex) and Fluorescein (Flu). In the below table 1 the amounts of drug, polymer, solvent, surfactant and type of surfactant and the aqueous medium are given.

The nanoparticles were prepared dissolving the PLGA-PTE 20K containing thioester bonds in acetone, miscible or partially miscible with water, subsequently the drug was added and dissolved or dispersed in it. Next the resulting organic solution was added to water containing a surfactant or surface active agent. The addition has been performed by injection. After the addition the mixture needs to be swirled by hand for 2 seconds to obtain proper homogenization. Hereafter the organic solvent has been removed by evaporation or extraction and the particles were dried.

Directly after the preparation of the nanoparticles the Z-average (hydrodynamic diameter) was measured in the Malvern Zetasizer Nano ZS.

| | | | | PLGA- | | | | |
|---|---|---|---|---|---|---|---|---|
| | Rapa | Dex | Flu | PTE | Acetone | Surfactant | H2O | Z-aver- |
| EX14 | mg | mg | mg | 20K mg | mL | mg | mL | age nm |
| A | 10.2 | | | 10.1 | 1000 | 50.1 PVA | 10 | 172.2 |
| B | | 10.85 | | 5.25 | 1000 | 50.1 PVA | 10 | 160.7 |
| C | | | 0.4 | 5 | 1000 | 50.2 | 10 | 123.8 |
| | | | | | | Pluronic | | |
| | | | | | | F68 | | |

1. Particles suitable for delivery of active agents comprising a polymer containing thioester bonds which are obtained via the reaction of a thioic acid functionality and an unsaturated group.

2. Particles according to claim 1 in which the polymer is obtained via the reaction of a component X comprising at least one ethylenically unsaturated group with a component Y comprising at least two thioic acids, wherein X and/or Y is a low molecular fragment, an oligomer or a polymer, whereby at least one of X or Y is an oligomer or a polymer and allowing the components to form a polymer comprising at least two thioester bonds.

3. Particles according to claim 1 wherein the polymer
comprises fragments of formula 3 and/or 4, 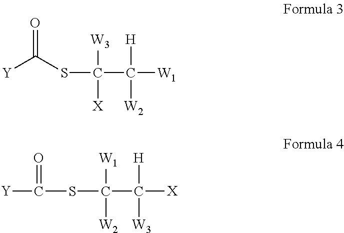
wherein
X and/or Y is a low molecular fragment, oligomer or a polymer whereby at least one of X or Y is an oligomer or polymer and whereby
W1, W2 and W3 are selected from the group consisting of C, H, O, N, S, P, alkyl, aryl, ester and ether.

4. Particles according to claim 1
wherein the polymer further
comprises a fragment according to formula 5, wherein
W1, W2 and W3 are selected from the group consisting of H, C, O, N, S, P, alkyl, aryl, ester and ether, and
wherein Y can be of a low molecular weight fragment, an oligomer or polymer, X can be the same or different low molecular weight fragment, oligomer or polymer wherein at least one of X or Y is an oligomer or polymer,
m and n are integers the sum of which indicates the number of thioester linkers connected to Y, wherein the sum of m and n is at least 2.

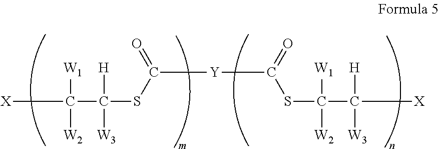

5. Particles according to claim 1 whereby X and/or Y are degradable.

6. Particles according to claim 1 wherein the average diameter is in the range of 10 nm to 1000 μm.

7. Particles according to claim 6, wherein the particles are microparticles with an average diameter in the range of 1 μm-1000 μm.

8. Particles according to claim 6, wherein the particles are nanoparticles with an average diameter in the range of 10 nm-less than 1 μm.

9. Particles according to claim 1 wherein the particles are provided with a structure comprising an inner core and an outer shell.

10. Particles according to claim 1 comprising one or more active agents.

11. Particles according to claim 10, wherein the active agent is selected from the group of nutrients, pharmaceuticals, proteins and peptides, vaccines, genetic materials, diagnostic agents or imaging agents.

12. Method for preparing particles according to claim 1 comprising the steps of
a) dissolving the polymer containing thioester bonds in at least one organic solvent, miscible or partially miscible with water,
b) subsequently adding a drug and dissolving or dispersing it,
c) addition of the resulting organic solution to an aqueous solution containing a surfactant or surface active agent and stirring it,
d) removing the organic solvent by evaporation or extraction,
e) drying the particles.

13. Method for preparing particles according to claim 1
comprising the steps of
a) dissolving the drug in an aqueous solution,
b) adding it to an organic solution containing the polymer containing thioester bonds,
c) mixing the resulting mixture
d) adding the resulting mixture to an aqueous solution containing a surfactant or a surface active agent and stirring it,
e) removing the an organic solvent by evaporation or extraction and
f) drying the particles.

14. Particles according to claim 1 for use in medical field.

15. Particles according to claim 1 for use in drug delivery.

16. Use of the particles according to claim 1 as a delivery system for a drug.

17. Use of the particles according to claim 1 in dermatology, muscular skeletal, oncology, vascular, orthopaedics, ophthalmic, spinal, intestinal, pulmonary, nasal, or auricular.

18. Use of the particles according to claim 1 in suspensions, capsules, tubes, pellets, (rapid prototyped) scaffolds, coatings, patches, aerosols, composite materials or plasters, (in situ forming) gels or aerosols.

19. Use of the particles according to claim 1 whereby the particles can be injected, sprayed, implanted or absorbed.

20. Use of the particles according to claim 1 for the manufacturing of a medicament useful in the treatment of a disease in mammal such as humans.
